(19) United States
(12) Patent Application Publication
(10) Pub. No.: US 2024/0358739 A1
(43) Pub. Date: Oct. 31, 2024

(54) COMPOSITION AND METHOD TO TREAT CACHEXIA

(71) Applicant: THE TRUSTEES OF INDIANA UNIVERSITY, Bloomington, IN (US)

(72) Inventors: Andrea BONETTO, Denver, CO (US); Joshua Robert HUOT, Indianapolis, IN (US)

(21) Appl. No.: 18/688,475

(22) PCT Filed: Sep. 23, 2022
(86) PCT No.: PCT/US2022/076923

§ 371 Date: Mar. 1, 2024

Related U.S. Application Data
(60) Provisional application No. 63/248,128, filed on Sep. 24, 2021.

(51) Int. Cl. A61K 31/713; A61P 35/00; C07K 16/22; C12N 15/113; G01N 33/574

(52) U.S. Cl. CPC A61K 31/713; A61P 35/00; C07K 16/22; C12N 15/113; G01N 33/57419; C07K 2317/76; C12N 2310/141; G01N 2333/4745

(57) ABSTRACT

Colorectal cancer (CRC) is often accompanied by cachexia, a multi-organ wasting syndrome which compromises musculoskeletal health. The disclosure is directed to the discovery that elevated levels of insulin-like growth factor binding protein 1 (IGFBP1) associated with cancers such as colorectal cancer (CRC) does not result from direct production of IGFBP1 by cancer cells. Methods and compositions to prevent or reduce the effects of cachexia are provided wherein a subject at risk of cachexia is administered a pharmaceutical composition comprising an inhibitor of IGFBP1.

### CROSS REFERENCE TO RELATED APPLICATIONS

This application claims priority to the following: U.S. Provisional Patent Application No. 63/248,128 filed on Sep. 24, 2021, the disclosure of which is expressly incorporated herein.

### GOVERNMENT LICENSE RIGHTS

This invention was made with government support under AR079379 awarded by National Institutes of Health. The Government has certain rights in the invention.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 6 kilobytes xml file named “29920-366106.xml,” created on Aug. 3, 2022.

### BACKGROUND

Colorectal cancer (CRC) is often accompanied by cachexia, a multi-organ wasting syndrome which compromises musculoskeletal health. In preclinical studies, we and others have shown that CRC is accompanied by metabolic and genomic perturbations of the liver, and further, that CRC liver metastases (LM) exacerbate musculoskeletal wasting. These observations, along with evidence indicating that liver-derived factors (i.e., hepatokines) may poorly influence musculoskeletal health across several disease states, suggest that the liver may play a role in mediating cancer-induced cachexia. A need exists to provide a composition and method of treatment to prevent or reduce the effects of cachexia in subjects having colorectal cancer.

### SUMMARY

The disclosure is directed to the discovery that elevated levels of insulin-like growth factor binding protein 1 (IGFBP1) associated with cancers such as colorectal cancer (CRC) does not result from direct production of IGFBP1 by cancer cells. Instead, applicant has discovered that CRC cells instigate liver cells to begin expressing IGFBP1. Further, it is shown that IGFBP1 plays a role in muscle and bone wasting (i.e., cachexia) in CRC subjects. In accordance with one embodiment compositions are provided comprising compounds that decreases the activity of IGFBP1 either by interfering with the expression of IGFBP1 or preventing IGFBP1 from interacting with its native ligand. In one embodiment the methods of administering those compositions, are provided to prevent or reduce the effects of cachexia in a subject.

In accordance with one embodiment a method of treating cachexia in a subject is provided. In one embodiment the subject is a cancer patient experiencing muscle loss and/or bone loss, or a cancer patient determined to be at risk for developing cachexia. In one embodiment the patient has been diagnosed with colorectal cancer. In one embodiment the method comprises administering to the patient a therapeutically effective amount of an inhibitor of IGFBP1, wherein said inhibitor decreases the expression of IGFBP1 or decreases the activity of IGFBP1 (e.g., by interfering with IGFBP1 binding to its native ligand or stimulating the destruction or removal of IGFBP1). In one embodiment the inhibitor of IGFBP1 is an antibody or a small molecule antagonist of IGFBP1. In one embodiment the inhibitor is an interference oligomer that binds to:
i) a nucleobase sequence of
atgtcagagg tccccgttgc tcgcgtctgg ctggtactgc tcctgctgac tgtccaggtc ggcgtgacag ceggcgctcc gtggcagtgc gegccctgct cegccgagaa getegegctc tgcccgccgg tgtccgcctc gtgcteggag gtcacceggt cegccggctg cggctgttgc ccgatgtgeg ccctgcctct gggcgcegcg tgeggcgtgg cgactgcacg ctgcgcccgg ggactcagtt gccgegcgct gccgggggag cagcaaccte tgcacgccct cacccgcggc caaggcgcct gegtgcagga gtctgacgcc tccgctcccc atgctgcaga ggcagggagc cctgaaagcc cagagagcac ggagataact gaggaggagc tcctggataa tttccatctg atggcccctt ctgaagagga tcattccatc ctttgggacg ccatcagtac ctatgatggc tcgaaggctc tccatgtcac caacatcaaa aaatggaagg agccctgccg aatagaactc tacagagtcg tagagagttt agccaaggca caggagacat caggagaaga aatttccaaa ttttacctgc caaactgcaa caagaatgga ttttatcaca gcagacagtg tgagacatcc atggatggag aggcgggact ctgctggtgc gtctaccctt ggaatgggaa gaggatccct gggtctccag agatcagggg agaccccaac tgccagatat attttaatgt acaaaactag (SEQ ID NO: 2), or a complete complement thereof, or
ii) a contiguous 10, 15, 17, 20 or 25 bp or longer fragment sequence of SEQ ID NO: 2 or a corresponding complement thereof. In one embodiment the interference oligomer is a nucleic acid such as an siRNA, or an miRNA, or a nucleic acid analog including for example a morpholino. In one embodiment the IGFBP1 interference oligomer is delivered to the cytosol of the patient's cells, optionally to the liver cells of the patient, via a viral vector, including for example use of an AAV based vector.

In one embodiment IGFBP1 inhibitor is an interference oligonucleotide at least 8 nucleotides in length, wherein the oligonucleotide has at least 85%, 90%, 95% or 99% sequence identity to a continuous 8 nucleotide sequence of (SEQ ID NO: 2) or a complement thereof.

In one embodiment the interference oligonucleotide is an RNA comprising a locked nucleic acid, optionally wherein the locked nucleic acid is the N-terminal and/or C-terminal nucleotide in said interference oligonucleotide. In one embodiment a pharmaceutical composition is provided comprising oligonucleotide at least 8 nucleotides in length, wherein the oligonucleotide has at least 85%, 90%, 95% or 99% sequence identity to a continuous 8 nucleotide sequence of (SEQ ID NO: 2) or a complement thereof, and a pharmaceutically acceptable carrier.

### BRIEF DESCRIPTION OF THE DRAWINGS

FIG. 1 is an illustrative model of the effects of IGFBP1 bone, muscle, and liver in a subject having colorectal cancer (CRC).

FIG. 2A is a graph showing the circulating levels of IGFBP1 in plasma from CRC patients compared to control. The data in FIG. 2A was derived from running an ELISA on plasma from CRC and control patients.

FIG. 2B is a graph showing the survival probably in CRC with high vs. low IGFBP1. *p<0.05 vs. Control.

FIGS. 3A-3I: Characterization of mice bearing MC-38 tumors. FIGS. 3A & 3B show two graphs looking at skeletal muscle weight of Gastrocnemius (FIG. 3A) and Quadriceps (FIG. 3B) of control vs. MC38 tumor-bearing mice; FIG. 3C shows the plantar flexion in vivo muscle force in a control vs. MC38 tumor-bearing mice. FIG. 3D is a graph showing ex vivo EDL muscle contractility in a control vs. MC38 tumor-bearing mice. FIGS. 3E-3I shows 5 graphs looking at various measurements of bone histomorphometry (trabecular parameters including the bone volume fraction (Tb.BV/TV; FIG. 3E), thickness (Tb.Th; FIG. 3F), number (Tb.N; FIG. 3G), pattern factor (Tb.Pf; FIG. 3H) and separation (Tb.Sp; FIG. 3I)) determined for wild-type and IGFBP1KO mice implanted with MC38 metastatic tumors in control vs. four MC38 tumor-bearing mice (Tb.Th, TbN, Tb.pf and Tb.sp: *p<0.05, **p<0.01, ***p<0.001).

FIG. 4A shows a 3D reconstruction of trabecular bone in femurs from mice bearing subcutaneous (s.c.) and metastatic (met) C26 tumors. FIG. 4B is a graph showing the quantification of circulating IGFBP1 levels in the control, the C26 (s.c.) tumor-bearing mice, a sham surgery on the control, and the metastatic C26 (mC26) tumor-bearing mice. **p<0.01, ***p<0.001 vs. Control; $$p<0.01 vs. C26; ##p<0.01 vs. Sham.

FIG. 5 is a graph showing the quantified amount of IGFBP1 measured by ELISA in the plasma of male 8-week old MC38 tumor bearing mice, ApcMin mice, C26 (s.c.) mice, metastatic C26 mice (mC26), HCT116 host, and mHCT116 host.

FIG. 6. shows pre-osteoclast cultures (control and rmIGFBP1) differentiated into osteoclasts using the TRAP stain and a bar graph showing the number of osteoclasts counted in control culture vs. the rmIGFBP1 treated culture. (n=5). *p<0.05 vs. PBS

FIG. 7 shows pre-osteoclast cultures (control and anti-IGFBP1) co-cultured with AML12 cells previously exposed to CRC and treated with IGFBP1 antibody, differentiated into osteoclasts; and a bar graph showing the number of osteoclasts counted in the control-PBS vs. anti-IGFBP1 treated co-culture. (n=5). *p<0.01 vs. PBS.

FIGS. 8A-8E present data from MC-38 mice infected with AAV8-shIGFBP1 (FIGS. 8A-8D), and HCT116 CRC hosts administered anti-IGFBP1 antibodies (FIG. 8E-8I). FIGS. 8A-8D show bar graphs quantifying the amount of trabecular bone (measuring Tb.BV/TV; FIG. 8A, Tb.Th; FIG. 8B, Tb.sp; FIG. 8C, and TbN; FIG. 8D, respectively) remaining in femurs from MC38 tumor-bearing mice infected with AAV8-shIGFBP1, where *p<0.05, **p<0.01 vs. Control; $p<0.05, $$p<0.01 vs. MC-38 shScr. FIG. 8E presents the 3D images of the bones.

FIGS. 8F-8I show bar graphs quantifying the amount of trabecular bone (measuring Tb.BV/TV; FIG. 8F, Tb.Th; FIG. 8G, Tb.sp; FIG. 8H, and TbN; FIG. 8I, respectively) remaining in femurs of control mice, HCT116 CRC host mice, and HCT116 CRC host mice administered anti-IGFBP1 antibodies, where *p<0.05, **p<0.01 vs. Control; $p<0.05, $$p<0.01 vs. HCT116. FIG. 8J presents the 3D images of the bones.

FIG. 9 show C2C12 myotubes size in control cells (FIG. 9A) vs. C2C12 myotubes exposed to rmIGFBPR1 (FIG. 9B), and western blotting for ITGB1, pERK1/2/ERK1/2, pFAK/FAK (FIG. 9C) in cells exposed to rmIGFBPR1 vs control.

FIG. 10A shows western blotting for ITGB1, pERK1/2/ERK1/2, pFAK/FAK, MyHC in C2C12 myotube cells transfected with a scramble of siRNA or siTGB1 RNA. **p<0.01, ***p<0.001 vs. PBS; #p<0.05 vs. mC26.Fig. FIG. 10B present Western blots showing the expression levels of pFAK/FAK. ****p<0.001 vs. PBS in cells cultured in 5% plasma from an mC26 host without and without IGFBP1 antibody.

FIGS. 11A-11C show bar graphs comparing C26 hosts infected with AAV8-shIGFBP1 compared to AAV-shScr, measuring the circulating IGFBP1 (FIG. 11A); the weight of the quadriceps (FIG. 11B); and the in vivo plantarflexion force (FIG. 11C), produced by each group. *p<0.05 vs. AAV-shScr

FIG. 12A shows a bar graph quantifying the fold change in IGFBP1 mRNA expression in AML12 cells exposed to 3T3 cells (control) vs. AML12 cells exposed to C26 cells.

FIG. 12B is a bar graph showing mRNA expression levels fold changes of FGF21 or IGFBP1 in the cell lines AML12, MC38, C26, and HCT116. ***p<0.001 vs. AML12.

FIG. 12C is a bar graph showing the protein expression levels of IGFBP1 released into conditioned media by AML12 cells treated with PBS (control) or mFGF21.). *p<0.05, ***p<0.001 vs. control/AML12.

FIG. 13A & 13B show bar graphs measuring the level of IGFBP protein expression in conditioned media after 48 hours comparing to a transwell of CRC cells and AML12 (FIG. 13A) or a mixture of CRC and AML12 cells (FIG. 13B).

FIG. 13C is a bar graph showing the fold change in liver IGFBP1 mRNA in control mice compared to HCT116 (s.c) mice, sham surgery mice, and mHCT116 (metastatic). *p<0.05, ***p<0.001 vs. transwell/sham; ##p<0.01 vs. control; $$p<0.01 vs. HCT116.

FIG. 14 is a graph showing circulating IGFBP1 levels from wild-type and IGFBP1KO mice bearing MC38 liver metastases **p<0.01. 8-week-old C57BL/6J male wild-type and IGFBP1KO mice were intrasplenically (1.25×10⁵) injected with MC38 cells (n=5-7/group). Animals were sacrificed 19 days following tumor implantation and blood was collected for ELISA assessment of IGFBP1.

FIGS. 15A-15G present data from quantifications of trabecular bone in femurs from wild-type and IGFBP1KO mice carrying MC38 liver metastases. Post sacrifice, femur bones were assessed for parameters of trabecular bone by microcomputed tomography (μCT) as detailed in Example 1. FIG. 15A presents 3D reconstruction quantifications of trabecular bone *p<0.05, **p<0.01, wherein 8-week-old C57BL/6J male wild-type and IGFBP1KO mice were intrasplenically (1.25×10⁵) injected with MC38 cells (n=5-7/group). Animals were sacrificed 19 days following tumor implantation. FIGS. 15B-15F are graphs presenting trabecular parameters including the bone volume fraction (Tb.BV/TV; FIG. 15B), thickness (Tb.Th; FIG. 15C), separation (Tb.Sp; FIG. 15D), number (Tb.N; FIG. 15E), pattern factor (Tb.Pf; FIG. 15F) and connectivity density (Conn.Dn; FIG. 15G) determined for wild-type and IGFBP1KO mice implanted with MC38 metastatic tumors.

FIGS. 16A-16D provide graphs showing gastrocnemius weights (FIG. 16A), tibialis anterior weights (FIG. 16B), quadriceps weights (FIG. 16C) and in vivo plantarflexion force (FIG. 16D) from wild-type and IGFBP1KO mice bearing MC38 liver metastases *p<0.05, **p<0.01, ***p<0.001, ****p<0.0001. More particularly, 8-week-old C57BL/6J male wild-type and IGFBP1KO mice were intrasplenically (1.25×10⁵) injected with MC38 cells (n=5-7/group). Animals were sacrificed 19 days following tumor implantation and muscles were weighed.

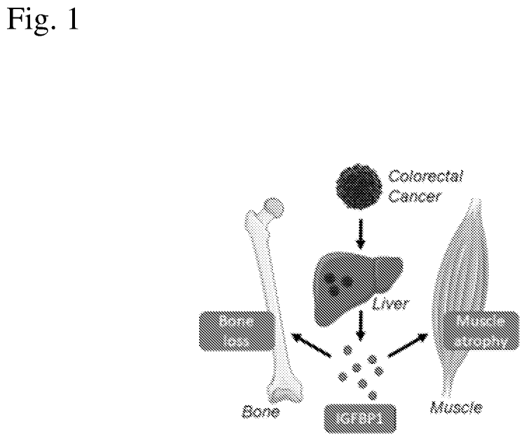

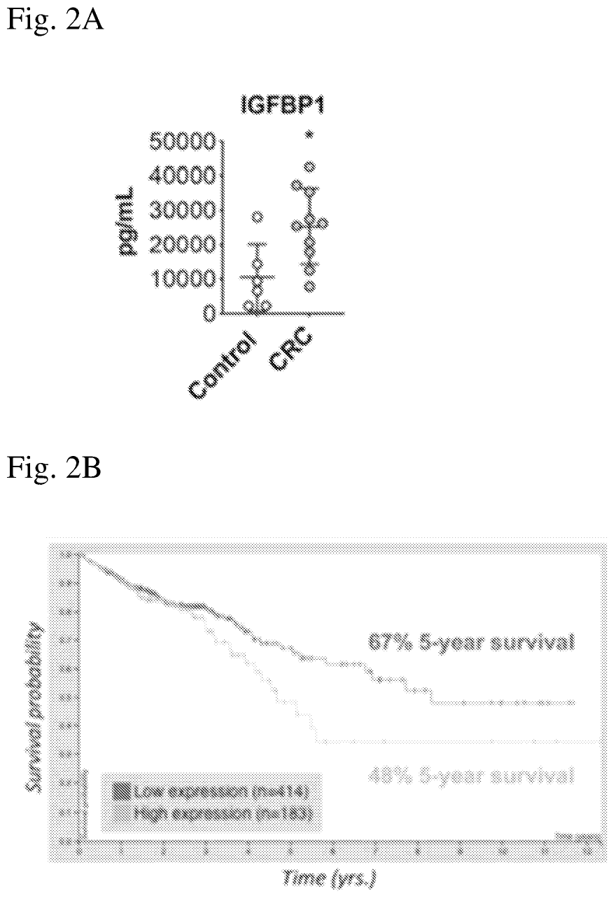

### DETAILED DESCRIPTION

Disclosed herein are compositions and methods for treating or prevent cachexia in a subject. The method includes administering a therapeutically effective amount of a composition that decreases the activity/level of IGFBP1 to a subject who is at risk of developing, is suspected of having, or is diagnosed with cancer cachexia.

### Abbreviations

IGFBP1 stands for insulin-like growth factor binding protein 1.

anti-IGFBP1 stands for an antibody or a portion of an antibody specific for IGFBP1.

rmIGFBP1 stands for recombinant IGFBP1

ITGB1 stands for integrin beta-1 receptor.

CRC stands for colorectal cancer.

LM stands for liver metastases.

C26 stands for colon-26 or adenocarcinoma cells that are used to model cachexia in animal models.

CM stands for conditioned media.

MC38 stands for murine colon adenocarcinoma.

HCT116 stands for human colorectal carcinoma cell line. When it is by itself in an image it may mean that the cells are injected into a mouse to form subcutaneous tumors.

mHCT116 stands for metastatic human colorectal carcinoma and is sometimes referred to as mHCT116 on graphs especially when comparing it to a HCT116 (s.c.).

C2C12 cells are myoblasts that can be differentiated into myotubes.

HSMM cells are human skeletal muscle myoblasts that can be differentiated into myotubes.

AML12 cells are healthy hepatocyte cells.

AAV stands for adeno-associated virus.

IGF1 stands for insulin-like growth factor 1.

IGF2 stands for insulin-like growth factor 2.

siRNA stands for small interfering RNA.

miRNA stands for micro RNA.

CRISPR stands for clusters of regularly interspaced short palindromic repeats.

Cas stands for CRISPR associated protein.

EDL stands for extensor digitorum longus.

ELISA stands for enzyme-linked immunosorbent assay.

ApcMin stands for Apc-multiple intestinal neoplasia—it is a point mutation introduced into mice to generate multiple intestinal adenomas.

mFGF21 is a recombinant protein of Fibroblast Growth Factor-21.

### Definitions

In describing and claiming the methods, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “treating” includes alleviation of the symptoms associated with a specific disorder or condition and/or preventing or eliminating said symptoms.

As used herein the terms “effective amount” or “therapeutically effective amount” of a compound refers to a nontoxic but sufficient amount of the compound to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein, unless specifically provided to the contrary, % and wt. % will equally mean % by weight of the total weight.

The term “parenteral” means not through the alimentary canal but by some other route such as intranasal, inhalation, subcutaneous, intramuscular, intraspinal, or intravenous.

As used herein, the term “purified” relates to the isolation of a molecule or compound in a form that is substantially free of contaminants normally associated with the molecule or compound in a native or natural environment and means having been increased in purity as a result of being separate from other components of the original composition.

As used herein the terms “maintain,” “prevent,” and “reduce the effects of” means to not lose up to about 5% or gain up to about 5% of tissue mass.

As used herein the term “subject” means an animal including but not limited to, humans, domesticated animals including horses, dogs, cats, cattle, and the like, rodents, reptiles, and amphibians

As used herein the term “patient” means an animal including but not limited to, humans, domesticated animals including horses, dogs, cats, cattle, and the like, rodents, reptiles, and amphibians being administered a therapeutic treatment either with or without physician oversight.

As used herein “Interfering RNA” is any RNA involved in post-transcriptional gene silencing, which definition includes, but is not limited to, double stranded RNA (dsRNA), small interfering RNA (siRNA), and microRNA (miRNA) that are comprised of sense and antisense strands.

As used herein a “locked nucleic acid” (LNA), is a modified RNA nucleotide in which the ribose moiety is modified with an extra bridge connecting the 2′ oxygen and 4′ carbon. For example, a locked nucleic acid sequence comprises a nucleotide of the structure:

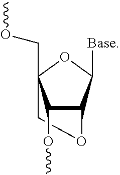

### EMBODIMENTS

As disclosed herein insulin-like growth factor binding protein 1 (IGFBP1), a liver-derived factor, has been identified as a potential mediator of musculoskeletal deficits in colorectal cancer (CRC). CRC patients and mice bearing CRC tumors (C26, MC38, HCT116, APC^{min}) consistently demonstrated markedly elevated circulating plasma IGFBP1, also supported by increased liver IGFBP1 mRNA expression in C26, MC38 and HCT116 hosts. Follow-up in vitro studies demonstrated that normal AML12 hepatocytes co-cultured with C26, MC38, and HCT116 CRC cells present elevated IGFBP1 secretion.

Of note, CRC cells lack IGFBP1 expression, thereby suggesting that IGFBP1 is host-derived. Treatment with recombinant IGFBP1 (rIGFBP1) was sufficient to promote differentiation of osteoclast precursor cells, while also promoting atrophy of C2C12 and HSMM myotubes, in line with activation of ITGB1 signaling, as reflected by increased ERK and FAK phosphorylation. Conversely, use of an IGFBP1 inhibitor (e.g., a neutralizing antibody specific for IGFBP1) reduced osteoclastogenesis and preserved C2C12 myotube size when exposed to serum from mice bearing C26 LM. Follow-up in vivo studies targeting IGFBP1 also proved efficacious in preserving musculoskeletal health. AAV8-mediated knockdown of liver IGFBP1 by tail vein injection was conducted utilizing a shRNA sequence CCGGGCTTGATACATGAAGCTGGTTCTCGAGAACCAGCTTCATGTATCAAGCTTT TTG (SEQ ID NO: 3) that targets the sequence GCTTGATACATGAAGCTGGTT (SEQ ID NO: 4). Administration of the AAV8-mediated knockdown IGFBP1 shRNA sequence preserved cortical and trabecular bone as well as muscle specific force in animals bearing subcutaneous MC38 tumors. Similarly, daily systemic administration of anti-IGFBP1 neutralizing antibodies (commercially available from R&D systems: catalog #: AF1240, with the source being Polyclonal Goat IgG) sustained trabecular bone and muscle force in mice bearing HCT116 LM. Altogether, the data indicates that IGFBP1 is a uniquely host-derived factor and a novel mediator of musculoskeletal deficits in cancer cachexia. As therapeutic interventions for cancer and cachexia often target tumor-associated factors, this work provides methods to counteract host-derived factors in the treatment of cancer-associated muscle and bone defects.

IGFBP1—a liver-derived hormone belonging to the insulin-like growth factor family of binding proteins (IGFBPs)—was reported to promote osteoclastogenesis and bone resorption by binding to the integrin beta-1 (ITGB1) receptor on the osteoclast surface, and was found associated with reduced muscle mass in disease states. IGFBP1 was also previously described to participate in tumor initiation and metastasization. As disclosed herein, IGFBP1 directly contributes to the multi-organ wasting observed in colorectal cancer (CRC) cachexia. We detected high IGFBP1 circulating levels in CRC patients. Recombinant IGFBP1 promoted myotube atrophy and osteoclast differentiation. Mice bearing MC-38 CRCs displayed muscle and bone loss, along with markedly elevated circulating IGFBP1 and increased muscle ITGB1. Mice bearing subcutaneous C26 CRCs exhibited muscle atrophy without bone loss, whereas mice bearing C26 LMs demonstrated exacerbated musculoskeletal wasting, along with dramatically elevated IGFBP1. Anti-IGFBP1 antibodies prevented tumor-induced myofiber atrophy and osteoclastogenesis in cultures, while AAV-mediated depletion of liver IGFBP1 abolished bone loss and improved muscle wasting and muscle function in CRC hosts. IGFBP1 was also increased in hepatocyte-CRC mixed cultures and in the liver of animals with metastatic CRCs, in line with evidence suggesting a role of IGFBP1 in cancer growth and dissemination. Altogether, these observations suggest a role of IGFBP1 in CRC cachexia.

The blockade of IGFBP1 in myotube cultures and in mice bearing CRC prevents cancer-induced muscle wasting. Since IGFBP1 normally regulates the bioavailability of IGF1 and IGF2, known anabolic factors for muscle and bone, this could be simply due to correction of their circulating levels, which have been reported as reduced in cachexia. However, data from the literature suggests that IGFBP1 can bind to the transmembrane receptor ITGB1, thereby regulating osteoclast differentiation. Our preliminary findings suggest that ITGB1 is also present on the membrane of muscle cells, thus supporting the hypothesis that the same signaling pathway may regulate muscle size, as well.

Specific antibodies, portions of an antibody, antagonists such as small molecules, siRNA, miRNA, viral constructs etc. against IGFBP1 can be used in combination with other commonly prescribed therapeutics to prevent loss of muscle mass, muscle function, and bone mass in patients with cancer and potentially any other condition that causes muscle wasting. This would also contribute to a marked improvement of their quality of life, especially considering that preservation of muscle mass has been reported to play a critical role in improving overall survival and tolerability to chemotherapeutics in cancer. This would reduce weakness in the patient and allow them to return to normal after eradication of the tumor. It is possible that IGFBP1 is elevated also as a consequence of chemotherapy treatment.

IGFBP1 is elevated in CRC patients and animals bearing CRC tumors (either implanted subcutaneously or as advanced tumors accompanied by liver metastases). IGFBP1 is a liver-derived factor, which normally binds to IGF1 and IGF2, thereby regulating their bioavailability. Hence, we do not know if the beneficial effects on muscle and bone that we detected in in vitro and in vivo model of CRC-induced muscle and bone loss are simply a consequence of restored IGF-dependent anabolism. Based on data from the literature we know that IGFBP1 can bind to the transmembrane receptor ITGB1 on the surface of osteoclasts. Our initial findings suggest that IGFBP1 can also bind to ITGB1 on the surface of muscle cells. Our initial studies suggest that ITGB1 is also present on the membrane of muscle cells, thus supporting the idea that the same signaling pathway may regulate muscle size as well. In some embodiments, a method is provided comprising blocking IGFBP1 to prevent or reduce the musculoskeletal complications associated with CRC. Blocking or inhibiting IGFBP1 may be achieved by using specific neutralizing antibodies, portions of an antibody, one or more small molecule antagonists, siRNA, miRNA, or CRISPR Cas approaches.

In one Example, normal hepatocytes (AML12) were co-cultured with CRC cells and we observed elevated IGFBP1 levels in the culture media. We have assessed IGFBP1 circulating levels in CRC patients and at least 6 different CRC mouse models, and we have consistently found elevated IGFBP1. We have performed AAV-mediated IGFBP1 knock-down of IGFBP1 in CRC hosts and as a result observed preservation of both bone and muscle mass/function. We have also used anti-IGFBP1 neutralizing antibodies in association with in vitro and in vivo models and observed preservation of muscle and bone.

As disclosed herein compositions and methods are provided to treat cachexia to prevent or reverse loss of muscle and/or bone tissue in a patient, particularly a cancer patient or maintain existing tissues. In an illustrative aspect, a method of preventing or reducing the effects of cachexia in a subject comprises decreasing IGFBP1 activity in the patient in need thereof. Decreasing IGFBP1 activity can be accomplished by decreasing the amount of functional IGFBP1 protein in the cells of the patient by
i) altering/deleting the gene encoding IGFBP1;
ii) interfering with the transcription and/or translation of the IGFBP1 gene (e.g., administering an interfering RNA);
iii) modifying the IGFBP1 protein (e.g., binding to an anti-IGFBP1 antibody); or
iv) any combination of i)-iii).
   In one embodiment a method of preventing or reducing the effects of cachexia in a subject comprises administering an effective amount of a composition containing an inhibitor of IGFBP1 activity.

The IGFBP1 inhibitor may be selected from an antibody, a portion of an antibody, a small molecule antagonist, a combination of small molecule antagonists, siRNA, miRNA, CRISPR-Cas approaches, or a combination thereof. In accordance with one embodiment the expression of the IGFBP1 is decreased by transfecting the patient's cells, and more particularly, the liver cells of the patient, with an interference oligomer that targets the IGFBP1 gene or mRNA transcripts and comprises a sequence that specifically binds to the sequence of SEQ ID NO: 2 or its complement thereof, or comprises a contiguous 6, 8, 10, 15, 17, 20 or 25 bp or longer fragment sequence of SEQ ID NO: 2. In one embodiment the interference oligomer is an oligonucleotide, including for example an interference RNA, including for example a small interfering RNA (siRNA), or microRNA (miRNA). In one embodiment the interference oligomer is a modified DNA wherein the phosphodiester backbone of the native DNA has been replaced with a non-ionic mimetic. In one embodiment the interference oligomer is a phosphorodiamidate morpholino. In one embodiment the IGFBP1 inhibitor is an interference oligonucleotide at least 8 nucleotides in length, wherein the oligonucleotide has at least 95% or 99% sequence identity to a continuous 8 nucleotide sequence of (SEQ ID NO: 2) or a complement thereof. In one embodiment the IGFBP1 inhibitor comprises at least a 6, 8, 10, 15, 20, 25, 30 or 35 bp nucleotides fragment of (SEQ ID NO: 2) or a complement thereof. In one embodiment the interference oligonucleotide comprises a locked nucleic acid at the 5′ or 3′ end of the oligo nucleotide. In one embodiment the interference oligonucleotide is delivered to the cells of said patient via a viral vector.

The subject may be suspected of having or been diagnosed with colorectal carcinoma. In some aspects, the colorectal carcinoma has metastasized in the liver. The subject may be suspected of having or been diagnosed with a muscle wasting disease.

In order to prevent or reduce the effects of cachexia, the desired therapeutic effect of maintaining muscle tissue and/or bone tissue is measured by comparing before administration of the composition and after a scheduled administration plan. In some embodiments, the scheduled administration plan is one administration of any of the IGFBP1 inhibitor compositions disclosed herein. In some embodiments, the scheduled administration plan is more than one administration of the composition. In some embodiments, the scheduled administration plan is more than two administrations of the composition. In some embodiments, the composition is administered daily. In some embodiments, the composition is administered more than once daily. In some embodiments, the composition is administered every other day. In some embodiments, the composition is administered weekly. In some embodiments, the composition is administered monthly. The scheduled administration plan may be proscribed by a person having skill in the art. The scheduled administration plan is adjusted based on the subject and environment.

### Exemplified Embodiments

In accordance with embodiment 1, a method of treating cachexia in a subject is provided, wherein the method comprises administering a therapeutically effective amount of an inhibitor of IGFBP1.

In accordance with embodiment 2, the method of embodiment 1 is provided, wherein the inhibitor is an antibody specific to IGFBP1.

In accordance with embodiment 3, the method of embodiment 1 or 2 is provided, wherein an inhibitor of IGFBP1 is administered where the inhibitor is an interference oligomer that binds to
i) a nucleobase sequence of SEQ ID NO: 2, or a complete complement thereof, or
ii) a contiguous nucleotide fragment of at least 6, 8, 10, 15, 17, 20 or 25 bp of SEQ ID NO: 2 or a corresponding complement thereof.

In accordance with embodiment 4, the method of any one of embodiments 1-3 is provided, wherein an inhibitor of IGFBP1 is administered, wherein the inhibitor is an siRNA, or an miRNA or a combination thereof.

In accordance with embodiment 5, the method of any one of embodiments 3-4 is provided, wherein the target sequence of the siRNA or miRNA comprises the sequence of SEQ ID NO: 4.

In accordance with embodiment 6, the method of any one of embodiments 1-3 is provided, wherein an siRNA and/or miRNA inhibitor of IGFBP1 is administered, and the siRNA and/or miRNA comprises a nucleobase sequence having at least 95% sequence identity with SEQ ID NO: 3.

In accordance with embodiment 7, the method of any one of embodiments 1-6 is provided, wherein the subject is at risk of developing, is suspected of having, or is diagnosed with cachexia.

In accordance with embodiment 8, the method of any one of embodiments 1-7 is provided, wherein the subject has or is suspected of having metastatic colon cancer.

In accordance with embodiment 9, a method of preventing or reducing the effects of cachexia in a cancer patient is provided, said method comprising measuring IGFBP1 blood levels in said cancer patients to detect cancer patients with elevated IGFBP1 levels relative to disease free patients;
administering an inhibitor of IGFBP1 to said cancer patients having elevated IGFPB1 levels, to reduce IGFBP1 blood levels and prevent or reduce the effects of cachexia in said patients identified as having elevated IGFPB1 levels.

In accordance with embodiment 10, the method of embodiment 9 is provided, wherein the inhibitor is an antibody specific to IGFBP1.

In accordance with embodiment 11, the method of embodiment 9 or 10 is provided, wherein an inhibitor of IGFBP1 is administered where the inhibitor is an interference oligomer that binds to
i) a nucleobase sequence of SEQ ID NO: 2, or a complete complement thereof, or
ii) a contiguous nucleotide fragment of at least 6, 8, 10, 15, 17, 20 or 25 bp of SEQ ID NO: 2 or a corresponding complement thereof.

In accordance with embodiment 12, the method of any one of embodiments 9-11 is provided, wherein an inhibitor of IGFBP1 is administered, wherein the inhibitor is an siRNA, or an miRNA or a combination thereof.

In accordance with embodiment 13, the method of any one of embodiments 9-12 is provided, wherein the target sequence of the siRNA or miRNA comprises the sequence of SEQ ID NO: 4.

In accordance with embodiment 14, the method of any one of embodiments 9-13 is provided, wherein an siRNA and/or miRNA inhibitor of IGFBP1 is administered, and the siRNA and/or miRNA comprises a nucleobase sequence having at least 95% sequence identity with SEQ ID NO: 3.

In accordance with embodiment 15, the method of any one of embodiments 9-14 is provided, wherein an IGFBP1 inhibitor is administered wherein the IGFBP1 inhibitor is an interference oligonucleotide at least 8 nucleotides in length, wherein the oligonucleotide has at least 95% or 99% sequence identity to a continuous 8 nucleotide sequence of (SEQ ID NO: 2) or a complement thereof.

In accordance with embodiment 16, the method of any one of embodiments 9-15 is provided, wherein an IGFBP1 inhibitor is administered, wherein the IGFBP1 inhibitor is an siRNA, or an miRNA and the target sequence of the siRNA or miRNA comprises the sequence of SEQ ID NO: 4.

In accordance with embodiment 17, the method of any one of embodiments 9-15 is provided, wherein the IGFBP1 inhibitor is delivered to the cells of said patient via a viral vector.

### Example 1

### Characterization of Mice Bearing MC-38 Tumors

C57BL/6J (8-week old males; n=5/group) were injected with 1.0×10⁶ MC-38 CRC cells (in sterile PBS) or with empty vehicle (Sterile saline alone). Skeletal muscles (A) were harvested 4-weeks following tumor implantation and weighed. Plantarflexion force (B) was performed 24 hours prior to sacrifice.

### Brief Methodology for Plantarflexion Force:

Briefly, the left hind foot was positioned to align with the tibia at 90° and then taped into the footplate force transducer. The knee was clamped at the femoral condyles, and two disposable monopolar electrodes (Natus Neurology, Middleton, WI, USA) were placed subcutaneously posterior/medial to the knee in order to stimulate the tibial nerve. The peak twitch torque was first established in order to determine the maximal stimulus intensity, and mice were exposed to a stimulation of 0.2 ms at 100 Hz to determine the force output. At the time of sacrifice extensor digitorum longus (EDL) muscles underwent ex vivo muscle contractility (C)

### Brief Methodology for Ex Vivo Contractility:

EDL muscles were dissected from hind limbs, the tendons were tied to stainless steel hooks using 4-0 silk sutures, and the muscles were mounted between a force transducer (Aurora Scientific, Aurora, ON, Canada). The muscles were then immersed in a stimulation bath with O₂/CO₂ (95/5%) and Tyrode solution (121 mM NaCl, 5.0 mM KCl, 1.8 mM CaCl₂), 0.5 mM MgCl₂, 0.4 mM NaH₂PO₄, 24 mM NaHCO₃, 0.1 mM EDTA, and 5.5 mM glucose). The muscles were stimulated to contract using supramaximal stimuli between two platinum electrodes. Data were collected via the Dynamic Muscle Control/Data Acquisition (DMC) and Dynamic Muscle Control Data Analysis (DMA) programs (Aurora Scientific). Prior to each contraction bout, the muscle was lengthened to yield the maximum force (L0). The force-frequency relationships were determined via an incremental stimulation frequency protocol (0.5 ms pulses at 10, 25, 40, 60, 80, 100, 125 and 150 Hz for 350 ms at the supramaximal voltage) with 1 min rest periods between contractions. The muscle weight and L0 were used to determine the specific force.

Post sacrifice, femur bones were assessed for parameters of trabecular bone by microcomputed tomography (μCT).

### Methodology for μCT:

Following euthanasia, mouse carcasses were placed in 10% neutral buffered formalin and allowed to fix for 2 days. Following fixation, the carcasses were placed in 70% ethanol, and the right femurs were dissected and prepared for scanning on a high-throughput μCT specimen scanner. All the femur samples were rotated around their longitudinal axes, and images were attained using a Bruker Skyscan 1176 (Bruker, Kontich, Belgium) with an established set of parameters including pixel size=9 μm³; peak tube potential=50 kV; X-ray intensity=500 μA; and a 0.3° rotation step. The raw data files were then used to obtain a 3-dimensional cross-sectional image using the SkyScan reconstruction software (NRecon; Bruker, Kontich, Belgium). All the structural measures were calculated on the three-dimensional reconstructed images using the Skyscan CT Analyzer software (CTAn; Bruker, Kontich, Belgium). Trabecular bone was analyzed between 1.0 and 2.0 mm under the femoral distal growth plate using a threshold of 80-255. The trabecular parameters included the bone volume fraction (Tb.BV/TV), thickness (Tb.Th), separation (Tb.Sp), number (Tb.N), pattern factor (Tb.Pf) and connectivity density (Conn.Dn).

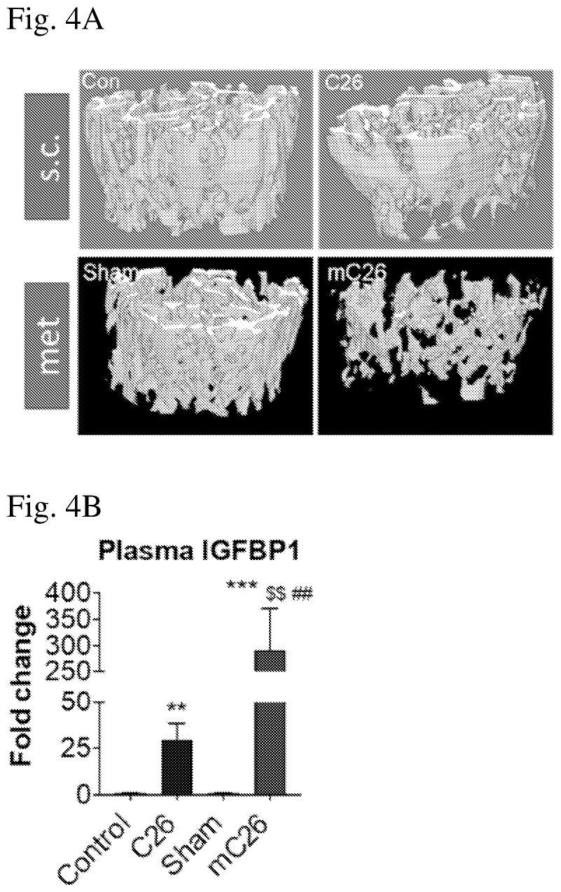

12-week-old CD2F1 male mice were either intrasplenically (mC26; 2.5×10⁵) or s.c. (C26; 1.0×10⁶) injected with C26 cells (n=4-6/group). Animals were sacrificed 2 weeks following tumor implantation. 3D reconstruction quantifications of trabecular bone in femurs from mice bearing subcutaneous (see FIG. 4A, top) and metastatic (see FIG. 4A, bottom) C26 tumors were obtained as described immediately above, under μCT. The circulating levels of IGFBP1 were obtained by ELISA on the serum from the tumor hosts (same tumor hosts as in FIG. 4A, and the results are shown in FIG. 4B. **p<0.01, ***p<0.001 vs. Control; $$p<0.01 vs. C26; ##p<0.01 vs. Sham.

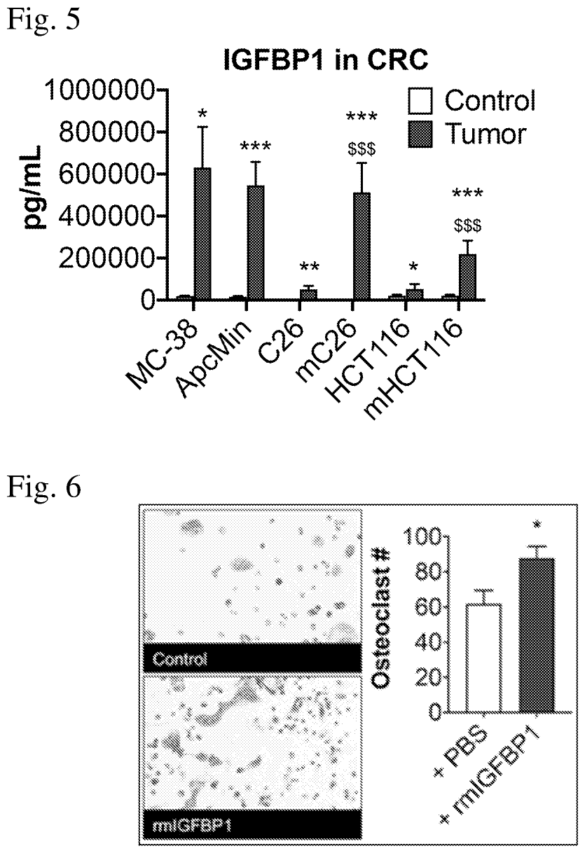

FIG. 5 is a graph of IGFBP1 levels measured by ELISA in the plasma of male 8-week old injected with MC-38, ApcMin, C26, mC26, HCT116 and mHCT116 hosts (n=5-8). All data in FIG. 5 was obtained by ELISA on serum from control and tumor hosts. More particularly,

C57BL/6J (8-week old males; n=5/group) were injected with 1.0×10⁶ MC-38 CRC cells (in sterile PBS) or with empty vehicle (Sterile saline alone). Blood was collected 4-weeks following tumor implantation.

ApcMin: Male Transgenic APCmin or WT littermates were sacrificed at 20 weeks of age, blood was collected.

C26 and mC26: 12-week-old CD2F1 male mice were either intrasplenically (mC26; 2.5×10⁵) or s.c. (C26; 1.0×10⁶) injected with C26 cells (n=4-6/group). Animals were sacrificed 2 weeks following tumor implantation, blood was collected.

HCT116 and mHCT116: 8-week old male NOD scid gamma (NSG; NOD-scid/IL2Rgnull) immunodeficient mice were enrolled into one of the following groups: subcutaneous injection of 3.0×10⁶ HCT116 tumor cells in sterile saline (HCT116, n=5) or an isovolumetric subcutaneous injection of vehicle (control, n=5); intrasplenic injection of 1.25×10⁵ HCT116 tumor cells in sterile saline (mHCT116, n=8) or an isovolumetric intrasplenic injection of vehicle (sham, n=5). Subcutaneous tumor hosts and experimental controls were sacrificed 30 days post tumor implantation, while mHCT116 and experimental controls (sham) were sacrificed 25 days post tumor implantation. Blood was drawn at the time of sacrifice.

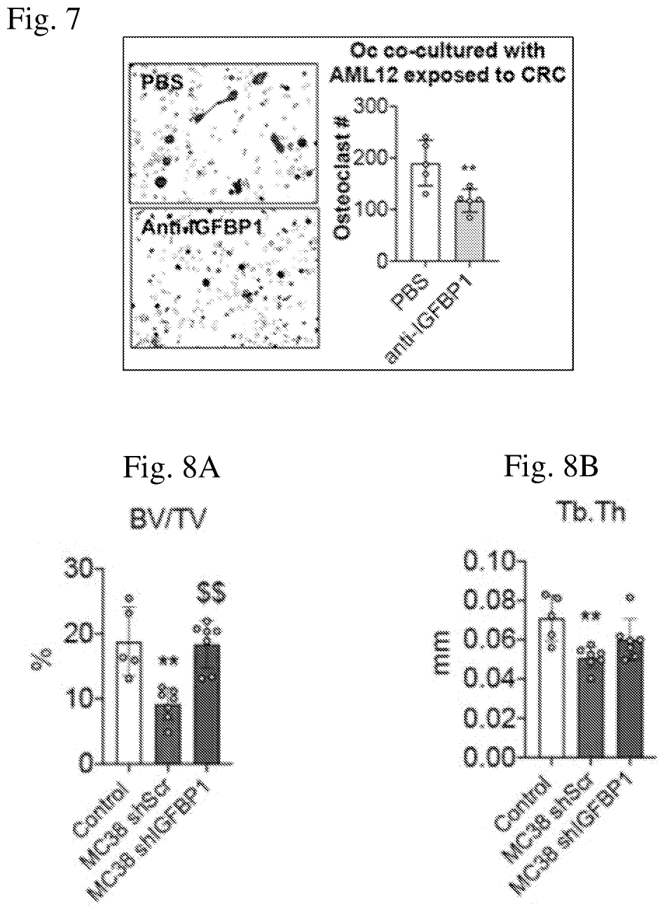

The quantification of osteoclast differentiation in pre-osteoclast cultures exposed to rmIGFBP1 and stained for TRAP (n=5) is shown in FIG. 6. Furthermore, quantification of osteoclast differentiation in pre-osteoclasts co-cultured with AML12 previously exposed to CRC and treated with anti-IGFBP1 antibody (n=5) is shown in FIG. 7. The osteoclast differentiation assay was conducted as follows:

Bone marrow cells were isolated from at least two 4-month-old wild-type male C57BL/6 mice by flushing the bone marrow out from both tibiae and femora with 10% FBS and 1% P/S-α-MEM. Cells were cultured for 48 h. Non-adherent cells were then collected and 14×10⁴ cells/cm2 were seeded on 96-well plates and cultured with RANKL 80 ng/ml (PeproTech) and M-CSF 20 ng/ml (PeproTech) added to induce osteoclast differentiation, the media was changed every 2 days. After 3 days of differentiation, 50 ng/mL of recombinant IGFBP1 or Vehicle (Control) was added to the osteoclast differentiation media for 48 h, after which cells were fixed for staining. Cells were stained using a TRAP kit (Sigma-Aldrich) and mature osteoclasts exhibiting 3 or more nuclei were quantified. Images were acquired using Axio Observer. Z1 motorized microscope (Zeiss, Oberkochen, Germany).

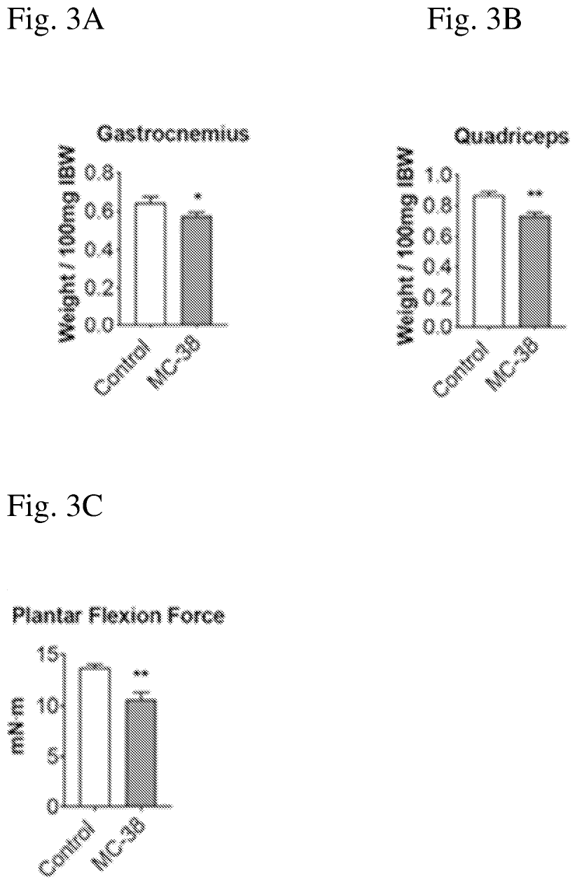

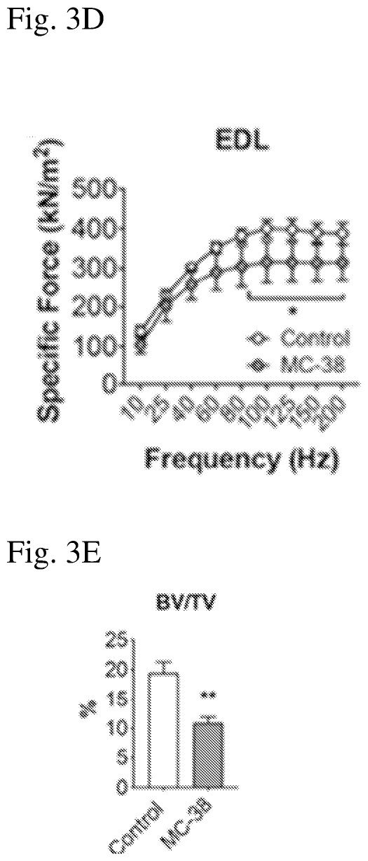

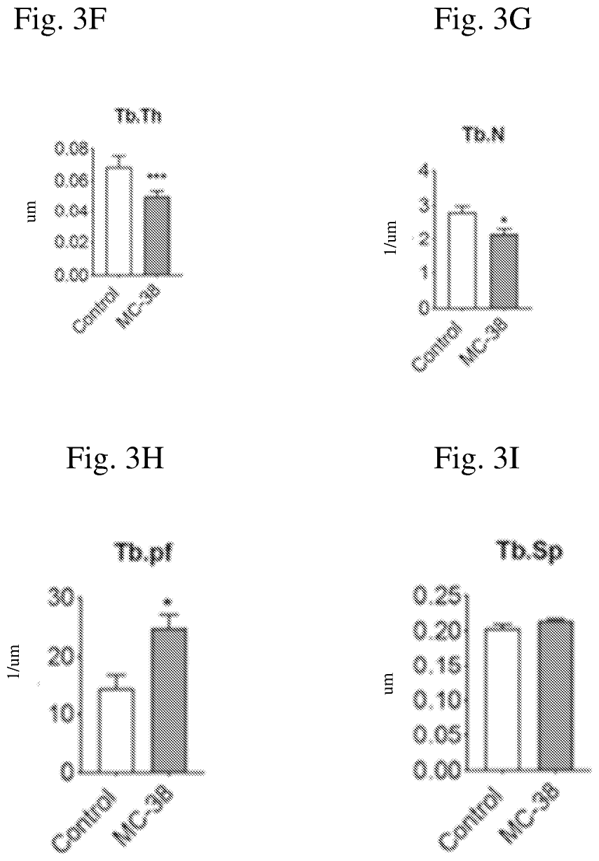

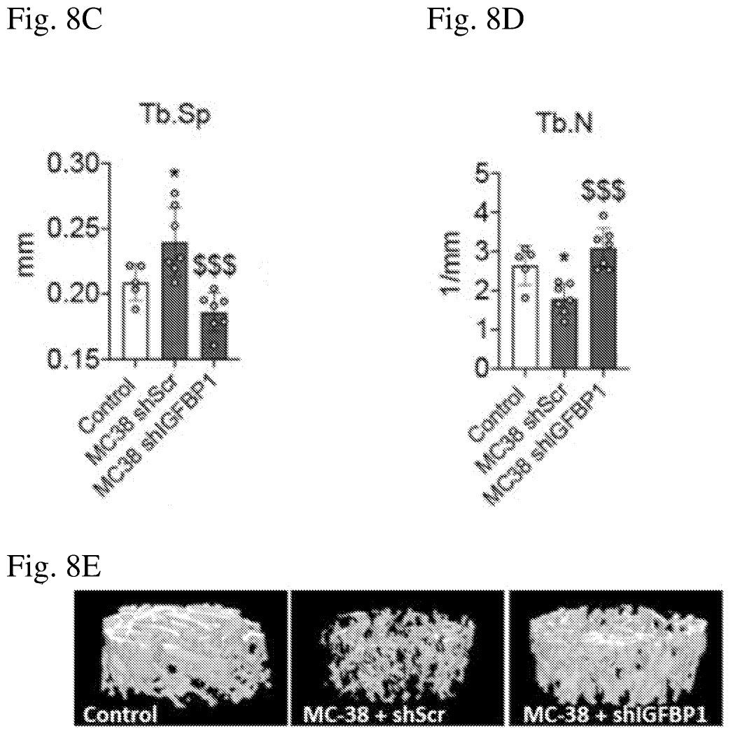

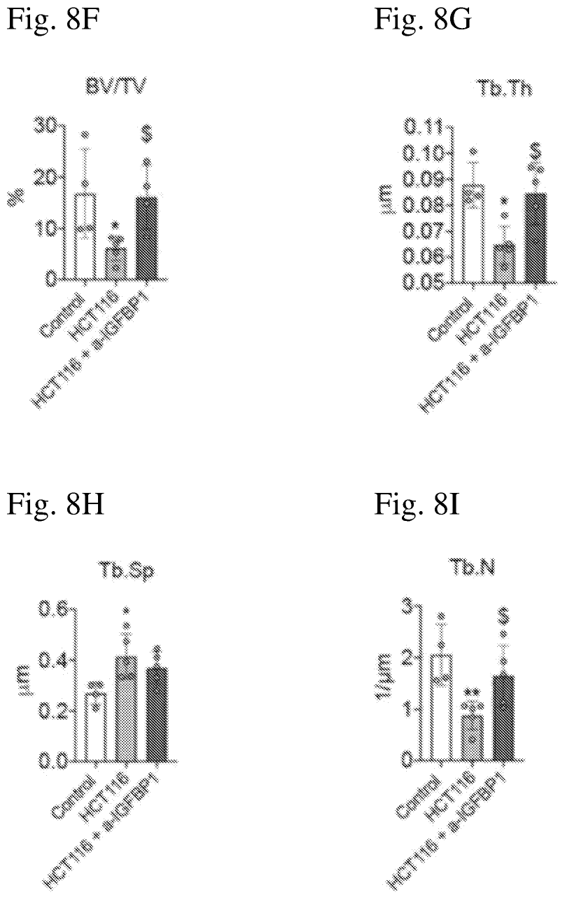

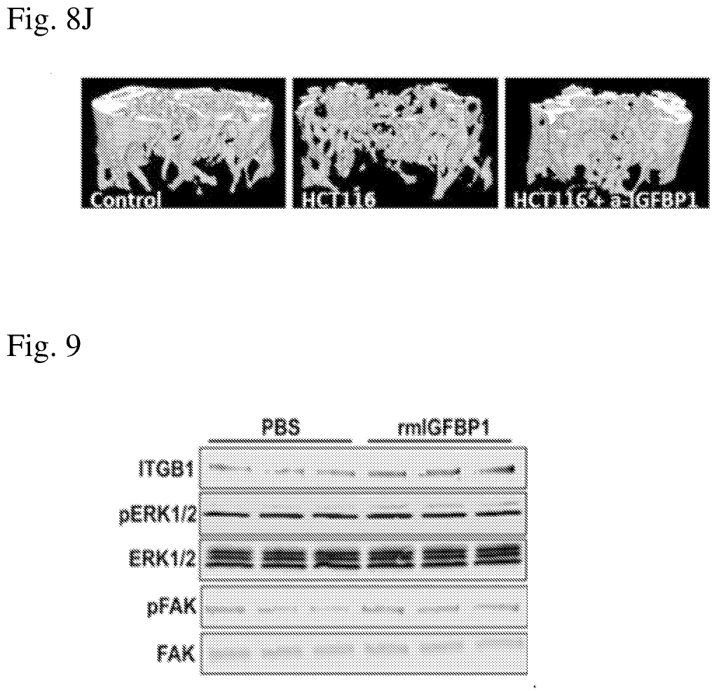

FIGS. 8A & 8B present data from 3D reconstruction quantifications of trabecular bone in femurs from MC-38 mice infected with AAV8-shIGFBP1 (FIG. 8A), and HCT116 CRC hosts administered anti-IGFBP1 antibodies (FIG. 8B). For MC-38 mice infected with AAV8-shIGFBP1, mice (C57BL/6J (8-week old males; n=5-8/group)) were subjected to a lateral tail vein injection of AAV8-shIGFBP1 (5×10¹² vg/mouse) or equal volume of AAV8-scramble. Following 2 weeks, mice were implanted with MC-38 cells (1.0×10⁶; subcutaneously) and were sacrificed 4 weeks later, using the same microCT methods as disclosed above for FIG. 3. For HCT116 CRC hosts administered anti-IGFBP1 antibodies, 8-week old male NOD scid gamma (NSG; NOD-scid/IL2Rgnull) immunodeficient mice were injected subcutaneously with 3.0×10⁶ HCT116 tumor cells in sterile saline (HCT116, n=5) or an isovolumetric subcutaneous injection of vehicle (control, n=5). 5 days following tumor transplantation mice were intraperitoneally injected with neutralizing antibodies against IGFBP1 (anti-IGFBP1: 0.03 mg/kg/day) or empty vehicle control every day until sacrifice (30 days), using the same microCT methods as disclosed above for FIGS. 3A-3I.

C2C12 myotubes were produced by exposing fully confluent myoblasts to DMEM containing 2% horse serum, 2 mM L-glutamine and 1% P/S, replacing the media every other day for 5 days. At 5 days post differentiation, myotubes were exposed to recombinant IGFBP1 (100 ng/ml) or empty vehicle control for 48 hours. FIG. 9 presents the results.

For myotube size: Differentiated C2C12 myotubes were fixed in ice-cold acetone: methanol for ten minutes, blocked for 1 h at room temperature and incubated overnight at 4° C. with an anti-myosin heavy chain antibody (MF-20, 1:100, Developmental Studies Hybridoma Bank). The following day, myotubes were incubated for 1 h at room temperature with an AlexaFluor 594-labeled secondary antibody (A11032, 1:500; Invitrogen). Myotubes were observed under an Axio Observer.Z1 motorized microscope (Zeiss) and analysis was performed by measuring the diameter of the narrowest portion along the multi-nucleate fibers (n=600 fibers per condition) using ImageJ software.

For western blots (FIG. 9): Cell layer protein extracts were obtained by homogenizing entire well surface in ice-cold RIPA buffer [150 mM NaCl, 1.0% NP-40, 0.5% sodium deoxycholate, 0.1% SDS and 50 mM Tris (pH 8.0)] supplemented with inhibitor cocktails for proteases (11697498001, Roche) and phosphatases (78428, Thermo Fisher Scientific). Following a 10-min incubation on ice, cellular debris was removed by centrifugation (15 min, 14,000 g at 4° C.), the supernatant was collected and protein concentration was assessed using the BCA protein assay method (Thermo Fisher Scientific). Protein extracts (30 μg) were electrophoresed in 4-15% gradient SDS Criterion TGX precast gels (Bio-Rad) and transferred to nitrocellulose membranes (30 min at 100 V; Bio-Rad). Following transfer, nitrocellulose membranes were blocked with odyssey blocking buffer (LI-COR Biosciences) at room temperature for 1 h, followed by an overnight incubation with gentle rocking with diluted primary antibody in odyssey blocking buffer containing 0.2% Tween-20 at 4° C. The following day, membranes were serially washed with PBS containing 0.2% Tween-20 (PBST) and the membrane was incubated at room temperature for 1 h with either anti-rabbit IgG (H+L) DyLight 800 or anti-mouse IgG (H+L) DyLight 680 secondary antibodies (1:10,000, 5151S and 5470S, Cell Signaling Technologies). Blots were again serially washed using PBST and then visualized and quantified using the Odyssey Infrared Imaging System (LI-COR Biosciences). Antibodies used were Integrin β1 (ITGB1) (1:1000, 34971), phospho-ERK1/2 (Thr202/Tyr204) (1:1000, #4370), ERK1/2 (1:1000, #4695), FAK (1:1000, #13009) from Cell Signaling Technologies; phospho-FAK Tyr 397 (1:1000, ab81298) from abcam.

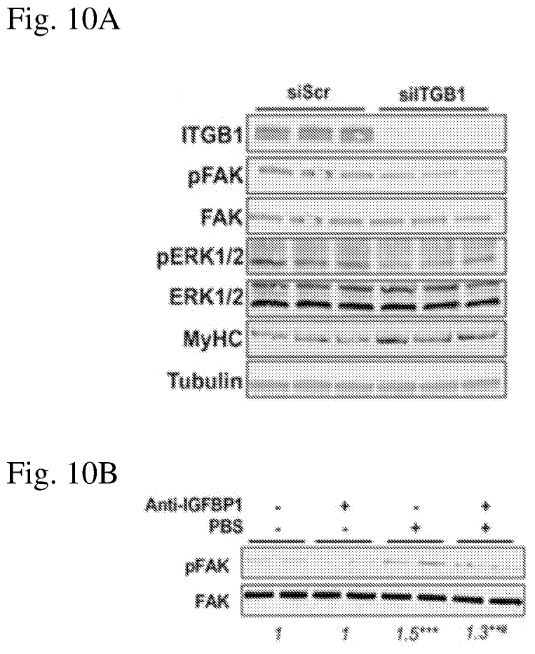

FIGS. 10A-10B presents data from myotube size in C2C12 cultures exposed to 5% plasma from mC26 hosts, with or without anti-IGFBP1 antibody.

C2C12 myotubes were produced by exposing fully confluent myoblasts to DMEM containing 2% horse serum, 2 mM L-glutamine and 1% P/S, replacing the media every other day for 5 days. At 5 days post differentiation, myotubes were exposed plasma conditioned media (5% from mC26 hosts) with or without neutralizing antibodies against IGFBP1 (100 ng/mL anti-IGFBP1 or vehicle) for 48 hours. Same procedures were used for staining of myotubes, and western blotting, as described for FIG. 9.

FIG. 10A & 10B: C2C12 myotubes were produced by exposing fully confluent myoblasts to DMEM containing 2% horse serum, 2 mM L-glutamine and 1% P/S, replacing the media every other day for 5 days. At 5 days post differentiation, myotubes were transfected with siRNAs for ITGB1 (25 pmol/ml). After 48 hours from transfection, myotubes were assessed for myotube size and protein expression (please see prior methods described for FIG. 9 for specifics). Note: α-Tubulin (1:1000, #12G10) from Developmental Studies Hybridoma Bank.

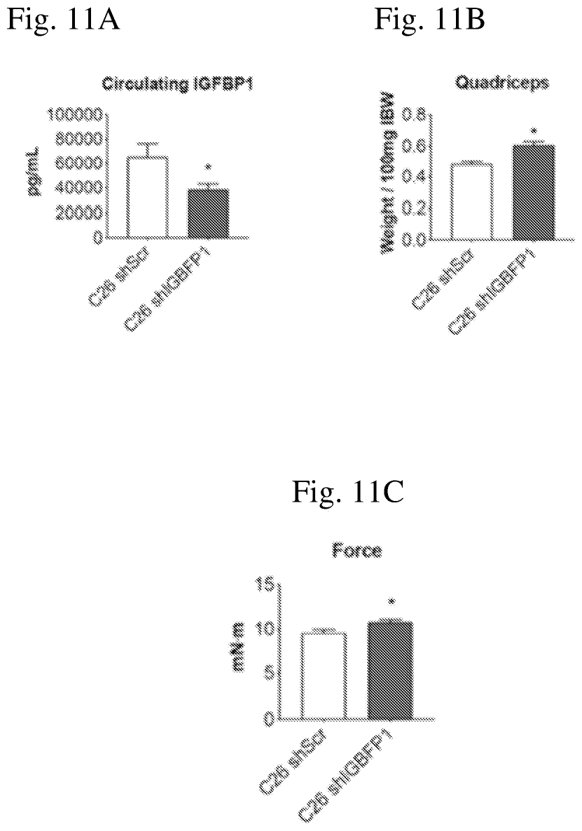

CD2F1 mice (8-week old males; n=4/group) were subjected to a lateral tail vein injection of AAV8-shIGFBP1 (5×10¹¹ vg/mouse) or equal volume of AAV8-scramble. Following 2 weeks, mice were implanted with C26 cells (1.0×10⁶; subcutaneously) and were sacrificed 2 weeks later. Blood (plasma) was collected for ELISA and quadriceps muscle was weighed. Results are presented in FIG. 11.

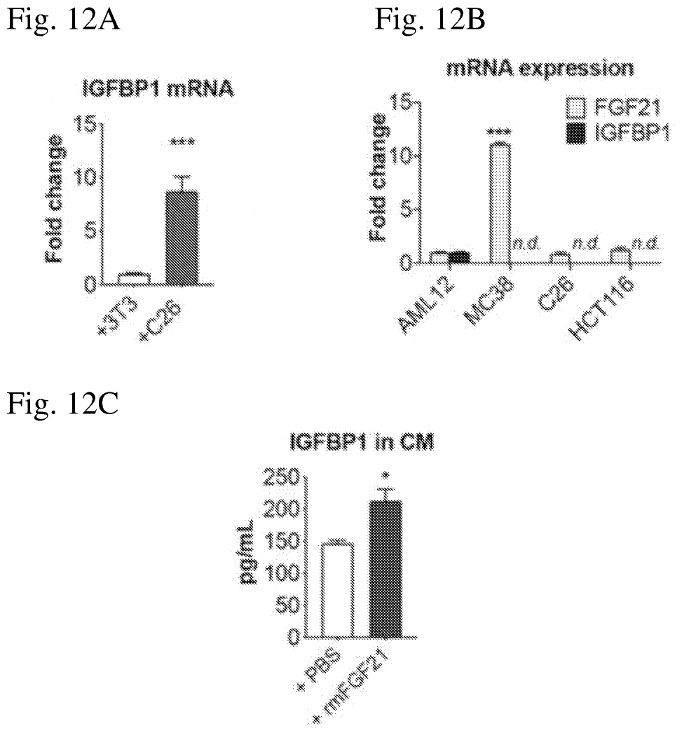

IGFBP1 mRNA expression in AML12 exposed to C26 cells (3T3 used as controls) (n=3) was measured and the results are presented in FIGS. 12A-12C. FIG. 12A: Fully confluent normal AML12 hepatocytes were co-cultured with 3T3 cells or C26 CRC cells for 48 hours. Following co-culture, RNA was extracted and gene expression for IGFBP1 was assessed using RT-qPCR. RNA methodology is outlined below. FIG. 12B: Normal AML12 hepatocytes, MC-38, C26, and HCT116 CRC cells were grown in 6-well plates to full confluence and RNA was extracted. FGF21 gene expression was assessed by RT-qPCR. RNA methodology is outlined below. FIG. 12C: Normal AML12 hepatocytes were grown to full confluence and treated with either recombinant FGF21 (100 ng/mL) or empty vehicle for 48 hours. Cell culture media was collected and assessed for IGFBP1 by ELISA.

### Methods for RNA Extraction:

Total RNA from the quadriceps muscle was isolated using the miRNeasy Mini kit (Qiagen) following the manufacturer's instructions. Following extraction, RNA was quantified using a Synergy H1 spectrophotometer (BioTek). Total RNA was reverse transcribed to cDNA using the Verso cDNA kit (Thermo Fisher Scientific). Transcript levels were measured by Real-Time PCR (Light Cycler 96, Roche) taking advantage of the TaqMan gene expression assay system (Life Technologies). Expression levels for IGFBP1 (Mm00515154_m1) and FGF21 (Mm00840165_g1) were detected. Gene expression was normalized to Tbp (Mm01277042_m1) levels using the standard 2-AACT methods.

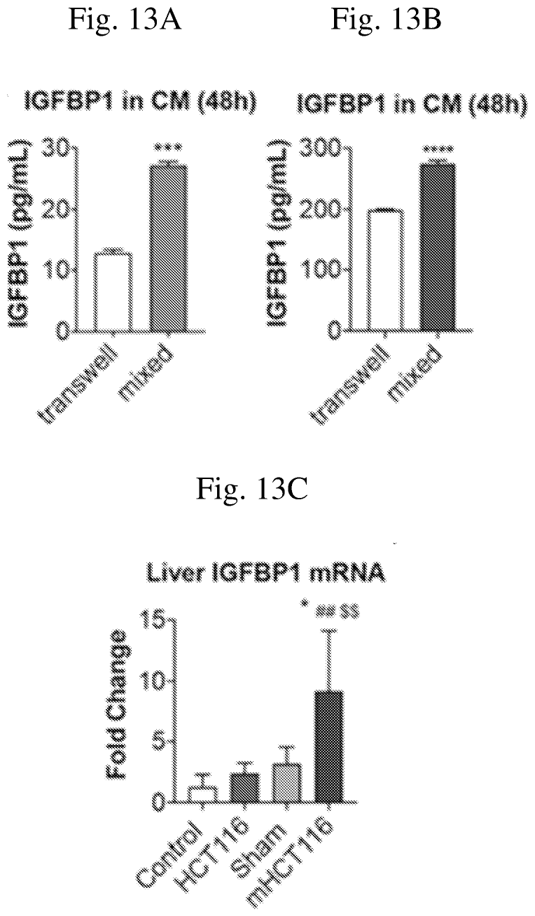

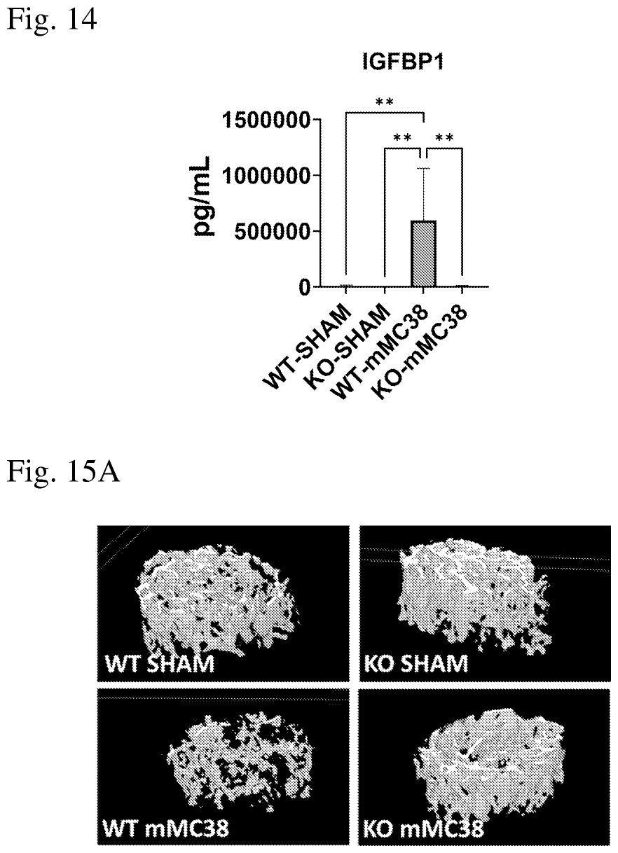

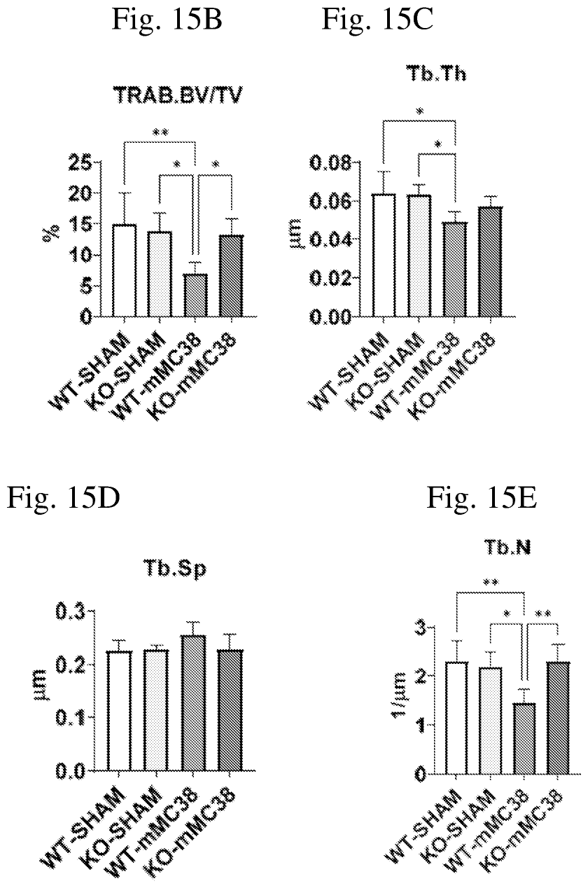

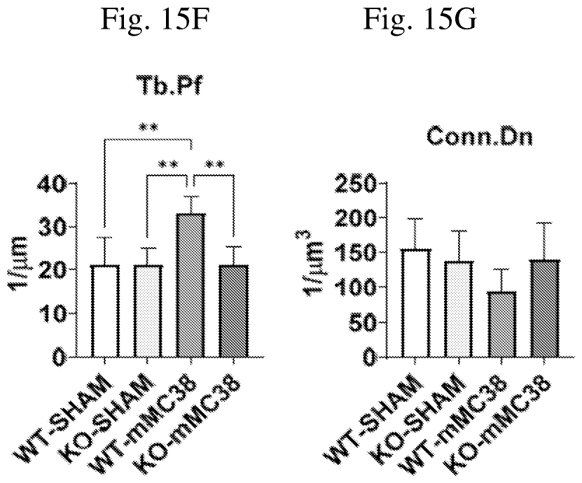

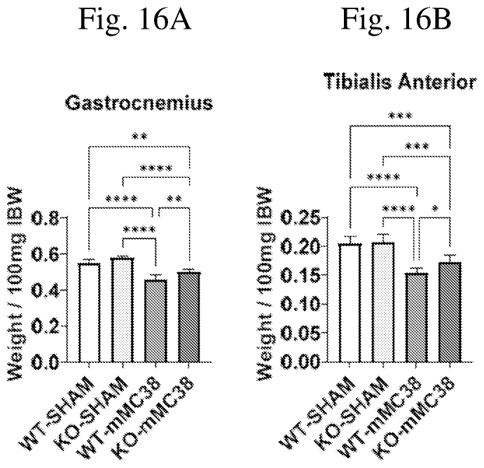

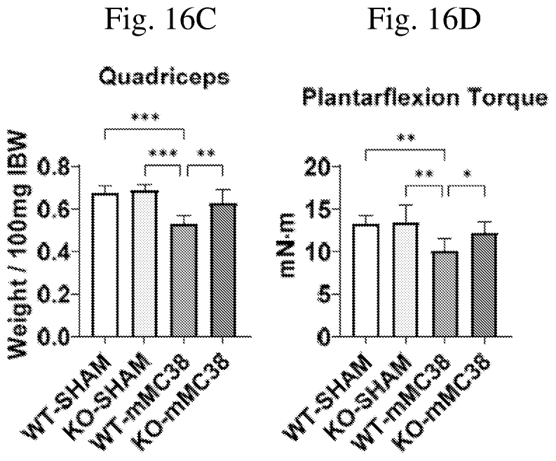

IGFBP1 levels in AML12 co-cultured with C26 or HCT116 cells (transwell or mixed; n=3) was measured and the results are presented in FIGS. 13A-13C.

FIG. 13A: Fully confluent normal AML12 hepatocytes were either co-cultured (using permeable transwell inserts) with C26 or HCT116 cells, or C26/HCT116 CRC cells were grown directly on top of the fully confluent AML12 cells. Co-culturing or mixed culturing took place for 48 hours. Cell culture media was collected for ELISA assessment of IGFBP1.

FIG. 13B: 8-week old male NOD scid gamma (NSG; NOD-scid/IL2Rgnull) immunodeficient mice were enrolled into one of the following groups: subcutaneous injection of 3.0×10⁶ HCT116 tumor cells in sterile saline (HCT116, n=5) or an isovolumetric subcutaneous injection of vehicle (control, n=5); intrasplenic injection of 1.25×10⁵ HCT116 tumor cells in sterile saline (mHCT116, n=10) or an isovolumetric intrasplenic injection of vehicle (sham, n=6). All animals were sacrificed 30 days post tumor implantation. Livers were processed for RNA extraction and subsequent gene expression of IGFBP1. See FIG. 12 notes for RNA extraction methods.

1. A method of treating cachexia in a subject, said method comprising administering a therapeutically effective amount of an inhibitor of IGFBP1.

2. The method of claim 1, wherein the inhibitor is an antibody specific to IGFBP1.

3. The method of claim 1, wherein the inhibitor is an interference oligomer that binds to
i) a nucleobase sequence of SEQ ID NO: 2, or a complete complement thereof, or
ii) a contiguous 10, 15, 17, 20 or 25 bp or longer fragment sequence of SEQ ID NO: 2 or a corresponding complement thereof.

4. The method of claim 1 wherein the inhibitor is an siRNA, or an miRNA.

5. The method of claim 4 wherein the target sequence of the siRNA or miRNA comprises the sequence of SEQ ID NO: 4.

6. The method of claim 5 wherein the siRNA or miRNA comprises a nucleobase sequence having at least 95% sequence identity with SEQ ID NO: 3.

7. The method of claim 1, wherein the subject is at risk of developing, is suspected of having, or is diagnosed with cachexia.

8. The method of claim 1, wherein the subject has or is suspected of having metastatic colon cancer.

9. A method of preventing or reducing the effects of cachexia in a cancer patient, said method comprising
measuring IGFBP1 blood levels in said cancer patients to detect cancer patients with elevated IGFBP1 levels relative to disease free patients;
administering an inhibitor of IGFBP1 to said cancer patients having elevated IGFPB1 levels, to reduce IGFBP1 blood levels and prevent or reduce the effects of cachexia in said patients identified as having elevated IGFPB1 levels.

10. The method of claim 9, wherein the inhibitor is an antibody specific to IGFBP1.

11. The method of claim 9, wherein the inhibitor is an interference oligomer that binds to
i) a nucleobase sequence of SEQ ID NO: 2, or a complete complement thereof, or
ii) a contiguous 10, 15, 17, 20 or 25 bp or longer fragment sequence of SEQ ID NO: 2 or a corresponding complement thereof.

12. The method of claim 9 wherein the IGFBP1 inhibitor is an interference oligonucleotide at least 8 nucleotides in length, wherein the oligonucleotide has at least 95% or 99% sequence identity to a continuous 8 nucleotide sequence of (SEQ ID NO: 2) or a complement thereof.

13. The method of claim 9 wherein the IGFBP1 inhibitor is an siRNA, or an miRNA and the target sequence of the siRNA or miRNA comprises the sequence of SEQ ID NO: 4.

14. The method of claim 12 wherein the IGFBP1 inhibitor is delivered to the cells of said patient via a viral vector.

15. The method of claim 13 wherein the IGFBP1 inhibitor is delivered to the cells of said patient via a viral vector.


SEQUENCE LISTING (4 sequences)

SEQ ID NO: 1 - protein, 259 aa, Homo sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 2 - DNA, 780 bp, Homo sapiens
  source 1..780: mol_type = other DNA
atgtcagaggtccccgttgctcgcgtctggctggtactgctcctgctgactgtccaggtcggcgtgacagccggcgctccgtggcagtgcgcgccctgctccgccgagaagctcgcgctctgcccgccggtgtccgcctcgtgctcggaggtcacccggtccgccggctgcggctgttgcccgatgtgcgccctgcctctgggcgccgcgtgcggcgtggcgactgcacgctgcgcccggggactcagttgccgcgcgctgccgggggagcagcaacctctgcacgccctcacccgcggccaaggcgcctgcgtgcaggagtctgacgcctccgctccccatgctgcagaggcagggagccctgaaagcccagagagcacggagataactgaggaggagctcctggataatttccatctgatggccccttctgaagaggatcattccatcctttgggacgccatcagtacctatgatggctcgaaggctctccatgtcaccaacatcaaaaaatggaaggagccctgccgaatagaactctacagagtcgtagagagtttagccaaggcacaggagacatcaggagaagaaatttccaaattttacctgccaaactgcaacaagaatggattttatcacagcagacagtgtgagacatccatggatggagaggcgggactctgctggtgcgtctacccttggaatgggaagaggatccctgggtctccagagatcaggggagaccccaactgccagatatattttaatgtacaaaactag

SEQ ID NO: 3 - DNA, 58 bp, Mus musculus
  source 1..58: mol_type = other DNA
ccgggcttgatacatgaagctggttctcgagaaccagcttcatgtatcaagctttttg

SEQ ID NO: 4 - DNA, 21 bp, Mus musculus
  source 1..21: mol_type = other DNA
gcttgatacatgaagctggtt